SEQUENCE LISTING (8 sequences)

SEQ ID NO: 1 - DNA, 1067 bp, Oryza sativa
ccacgcgtccgctcgccgccgtcgaaacccaaaatcttctcttcccgtacgtgagaagcgccaggtcgtcgtcgccgccatgggcgtggaggtgtgcgtcaaggccgccgtcggccacccggacacgctcggcgactgtccattctcgcagagggtgctgctgactctggaggagaagaaggtgccctacgagatgaagctcatcgacgtccagaacaagcccgactggtttctgaagatcagcccagaggggaaggtgcctgtgtttaacggtggtgatggcaaatggattcctgattctgatgtgatcactcaagtcattgaggagaagtacccaaccccgtctcttgtcacccctcctgagtatgcatcagtgggatcaaaaattttctcatgcttcacaacgttcttgaagagcaaggatccaaatgatggttcagagaaggcacttcttactgaactgcaggcactcgaggagcatctgaaagctcatggcccctttatcaacgggcagaacatttcagctgctgaccttagcctggcaccaaagctctaccatctccaggttgctctggagcatttcaaaggctggaagatcccggaagacctaaccaatgttcatgcttacacagaggctctgtttagccgcgaatctttcatcaagacgaaggcagctaaggagcacctgattgctggatgggcaccaaaagtgaatgcgtaagagcctgcccttatgctctggtgctgcttggacaccatgctgtttatctgatcggtccatgtcagtggtgggcactactactactcttgtgtagcttgggtgcatgattgggttggaataatgtagcctcatccgttgagtaccttgatatggttgttgcaagtgtgcactttttctatgaactatctcctgctggcttaagtcgaaaccgtgggtcggtttggccttatgttcaactaagagagtgcatatactgtaatggaacctttgctagtacaatatgttatatgaataatggagatgcagcctgcagctgctcttgcttggaaaaaaaaaaaaaaaaaaa

SEQ ID NO: 2 - DNA, 1564 bp, Brassica rapa
taacaacaactttctgaagatcgatcggataaaaaatggcggagaagagcttcaagtacatcatcctcggcggcggcgtctcagccggatacgcagctaaggagtttgctagtcaaggagttaaaccaggggaattggcagttatctccaaagaggcggtggctccttatgaacgtcctgctcttagcaagggctatttgtttcctgaaggggcggctagactcccaggtttccattgctgtgttggtagtggtggagaaaaactgcttcctgaatcatacaaacagaaagggattgagttgatactaagcacggagatagtgaaagcagatctcgctgccaagagtcttgtcagtgcagctggggatgtcttcaaatatgagactctcataattgcaactggctctactgttctgagattgactgattttggtgtgaaaggtgctgactctaagaatatcctctatctgagggagattgatgatgcagacaaagtggttgaagctattcaagcaaagaaaggtggaaaggctgtggttgttggtggaggctacattggtcttgagcttagtgcagctttaaggatcaacaattttgatgtcactatggttttccctgaaccctggtgcatgcctaggcttttcaccgccgacattgctgcgttctatgagacttactatacaaacaagggagtgaagatcattaaaggaactgtggcatctgggttcacagcacatccaaatggagaggtgaatgaagtacaactcaaggatggaaggtcgctagaagccgacattgtgatagttggagttggtgcaagaccattaacagccttattcaagggacaggtcgaagaagacaaaggtggaatcaagaccgatgcattcttcaaaacaagtgttcctgatgtttacgctgttggtgacgttgccactttccccttgaaaatgtatggagacatgagaagggtcgagcatgttgaccattctcgcaaatccgcagagcaagctgttaaggcgatcaaagcggctgagggaggcggagcggtggaggaatacgactacctcccattcttctactcccgctcgtttgatctctcatggcagttctatggagacaacgtaggagactctgtcttgtttggagacagcaatccatcaaacccgaaaccaagatttggagcgtattgggttcaagatggtaaagtggttggagcattcatggaaggaggtagtggtgacgagaacaaagccttggcgaaagtggccaaagctagacctgctgcagagagcctcgaggatcttaccaaacaaggcatctcatttgctgctaagatctgaggagaagaaagatgggagagtttaaactttttttctattctattacaataaaagattatgttaagcaatgtgcttgcacgtacatatgctgaaataagttgggtgtttgaatgaaacctgattggatacttgtaaacgctattcatcaaactagataatgcgattttatattgactgtggcacatgggattagccaaaaaaaaaaaaaaaaaaaaaaaaaaaaa

SEQ ID NO: 3 - DNA, 27 bp, Artificial Sequence
  primer
tgccttcatacgctatttatttgcttg

SEQ ID NO: 4 - DNA, 27 bp, Artificial Sequence
  primer
ccttgctcttcaagaacgttgtgaagc

SEQ ID NO: 5 - DNA, 21 bp, Artificial Sequence
  primer
ccgttaataaatggaccctgc

SEQ ID NO: 6 - DNA, 21 bp, Artificial Sequence
  primer
aagcgcaattttacagctgag

SEQ ID NO: 7 - DNA, 28 bp, Artificial Sequence
  primer
caatcaagcattctacttctattgcagc

SEQ ID NO: 8 - DNA, 27 bp, Artificial Sequence
  primer
caatctcagaacagtagagccagttgc

(19) United States
(12) Patent Application Publication
(10) Pub. No.: US 2014/0245493 A1
(43) Pub. Date: Aug. 28, 2014

(54) USE OF PLANT-DERIVED DHAR OR MDHAR GENE AS A MODULATOR FOR CROP YIELD AND ENVIRONMENTAL STRESS

(71) Applicants: Ho Sung Yoon, Daegu (KR); Il Sup Kim, Chungcheongbuk-do (KR); Young Saeng Kim, Gyeongsangnam-do (KR)

(72) Inventors: Ho Sung Yoon, Daegu (KR); Il Sup Kim, Chungcheongbuk-do (KR); Young Saeng Kim, Gyeongsangnam-do (KR)

(73) Assignee: KYUNGPOOK NATIONAL UNIVERSITY INDUSTRY-ACADEMIC COOPEERATION FOUNDATION, Daegu (KR)

(21) Appl. No.: 14/241,014

(22) PCT Filed: Feb. 7, 2012
(86) PCT No.: PCT/KR2012/000864

§ 371 Date: Feb. 25, 2014

(30) Foreign Application Priority Data
Aug. 25, 2011 (KR) 10-2011-0085201
Aug. 25, 2011 (KR) 10-2011-0085204

(51) Int. Cl. C12N 15/82; A01H 5/10; C12N 9/02

(52) U.S. Cl. CPC C12N 15/8261; C12N 9/0051; C12N 15/8273; A01H 5/10
USPC 800/289; 435/468; 536/23.2; 800/290; 800/298

(57) ABSTRACT

The present invention relates to a method for increasing crop yield or enhancing resistance of a plant to environmental stress by using dhar (dehydroascorbate reductase) gene derived from rice (Oryza sativa) or mdhar (monodehydroascorbate reductase) derived from Chinese cabbage (Brassica rapa), a method of producing a transgenic plant with increased crop yield or enhanced resistance to environmental stress by using dhar gene or mdhar gene, a transgenic plant with increased crop yield or enhanced resistance to environmental stress that is produced by the aforementioned method, and a seed thereof, and a composition for increasing crop yield or enhanced resistance to environmental stress containing dhar gene or mdhar gene.

### CROSS REFERENCE TO RELATED APPLICATIONS AND CLAIM OF PRIORITY

This patent application is a National Phase application under 35 U.S.C. §371 of International Application No. PCT/KR2012/000864, filed 7 Feb. 2012, which claims priority to Korean Patent Application Nos. 10-2011-008520 filed 25 Aug. 2011, and 10-2011-0085204 filed 25 Aug. 2011, entire contents of which are incorporated herein by reference.

### BACKGROUND

1. Technical Field

The present invention relates to a use of dhar gene derived from rice or mdhar gene derived from Chinese cabbage as a modulator for crop yield and environmental stress. More specifically, it relates to a method for increasing plant crop yield by using dhar (dehydroascorbate reductase) gene derived from rice (Oryza sativa) or mdhar (monodehydroascorbate reductase) gene derived from Chinese cabbage (Brassica rapa), a method of producing a transgenic plant with increased crop yield by using rice-derived dhar gene or Chinese cabbage-derived mdhar gene, a transgenic plant with increased crop yield that is produced by the aforementioned method, and a seed thereof, a composition for increasing plant crop yield containing rice-derived dhar gene or Chinese cabbage-derived mdhar gene, a method for enhancing resistance of a plant to environmental stress by using rice-derived dhar gene or Chinese cabbage-derived mdhar gene, a method of producing a transgenic plant with enhanced resistance to environmental stress by using rice-derived dhar gene or Chinese cabbage-derived mdhar gene, a transgenic plant with enhanced resistance to environmental stress that is produced by the aforementioned method, and a seed thereof, and a composition for enhancing resistance of a plant to environmental stress containing rice-derived dhar gene or Chinese cabbage-derived mdhar gene.

2. Description of the Related Art

Due to increased income and improved living standards, people are now enjoying healthier and richer life than ever before. In response to those changes, it is required to develop food resources which can satisfy consumer's diverse tastes. In particular, both the farming families and consumers need to be satisfied with production of a high quality variety having good taste, nutritional value, or the like. Recently, genome sequence of rice was identified, and thus a base for analyzing the function of various rice genes has been established. Accordingly, it is necessary to develop a technique for isolating useful genes and producing good rice varieties satisfying the current requirement by using it.

Fortunately, thanks to recent sequencing of rice genome, most genes were identified and an insertional mutant population allowing easier analysis of those genes was constructed. Accordingly, there is a great need to have a technique for producing good rice varieties by utilizing such resource and analyzing and utilizing the function of useful genes.

Regarding the genome sequence analysis of rice, Rice Genome Program has been established and carried out mainly by Japan (International Rice Genome Sequencing Projects 2005). As the sequence analysis and annotation are completed, there is a fierce competition regarding the gene function analysis. Meanwhile, as rice is cultured mostly in Asia, the study relating to rice is mainly conducted in Asia containing mainly Japan, China and Korea. In Japan, in particular, systematic nation-level studies are carried out to analyze the gene function based on cDNA library construction and genome-wide full length cDNA ectopic expression. Highly competent labs in US, Europe, and Australia also conduct a research on rice, and that is because, after studying main disease-related characters by using rice as a model system, the results can be desirably applied to wheat, corn, soybean, etc. China lags behind Japan, but in terms of scale, it has superiority and started to conduct a large-scale study on function of rice genes.

In Korea, the study on function of rice genes has started long time ago, and the insertional mutant population has been produced by using T-DNA and Ac/Ds system. There are more than 100,000 T-DNA inserts which have been developed until now and, as there are more than 50,000 Ds-inserts established by Gyeongsang National University and Rural Development Administration of Korea, and all taken together, an insertional mutant has been established for most of the rice genes (Jeong et al. 2006 Plant J 45: 123-132). Those insertional mutants allow easier isolation of many gene variants based on an analysis of the sequence near insertion of T-DNA or Ds. Many labs in foreign countries also conduct similar studies, but they are yet to match the level of Korea. Meanwhile, it is considered that the techniques relating to rice transformation, culture, breeding or the like in Korea are not behind any of those in other countries all over the world. In fact, they are evaluated to be better than most of other countries. In particular, compared to Japan, US, and European countries in which strong restrictions are applied to a transformant, it is believed that Korea has a priority over them in terms of a basis needed for producing excellent rice varieties.

In Korean Patent Application Publication No. 2009-0119884, “A plant having character relating to enhanced crop yield and a method for producing the same” is disclosed. In Korean Patent Application Publication No. 2009-0027219, “A plant having character relating to enhanced crop yield with controlled expression of NAC transcription factor and a method for producing the same” is disclosed.

### SUMMARY

The present invention is devised under the circumstances described above, and the inventors of the present invention completed the present invention by confirming that crop yield of a transgenic rice plant is increased and resistance to environmental stress of a plant is enhanced by introduction of rice-derived dhar gene or Chinese cabbage-derived mdhar gene.

To solve the problems described above, the present invention provides a method for increasing plant crop yield by using dhar (dehydroascorbate reductase) gene derived from rice (Oryza sativa) or mdhar (monodehydroascorbate reductase) derived from Chinese cabbage (Brassica rapa).

The present further provide a method of producing a transgenic plant with increased crop yield by using rice-derived dhar gene or Chinese cabbage-derived mdhar gene.

The present further provide a transgenic plant with increased crop yield that is produced by the aforementioned method, and a seed thereof.

The present further provide a composition for increasing crop yield of a plant containing rice-derived dhar gene or Chinese cabbage-derived mdhar gene.

The present further provide a method for enhancing resistance of a plant to environmental stress by using rice-derived dhar gene or Chinese cabbage-derived mdhar gene.

The present further provide a method of producing a transgenic plant with enhanced resistance to environmental stress by using rice-derived dhar gene or Chinese cabbage-derived mdhar gene.

The present further provide a transgenic plant with enhanced resistance to environmental stress that is produced by the aforementioned method, and a seed thereof.

The present still further provide a composition for enhancing resistance of a plant to environmental stress containing rice-derived dhar gene or Chinese cabbage-derived mdhar gene.

The rice plant transformed with rice-derived dhar gene or Chinese cabbage-derived mdhar gene as developed by the present invention is characterized in that it has enhanced resistance to environmental stress and increased crop yield, and thus it can be very advantageously used for increasing crop yield even under the unfavorable conditions for culturing a rice plant.

### BRIEF DESCRIPTION OF THE DRAWINGS

FIG. 1 illustrates PCR isolation for selecting homozygote lines of T₂ transgenic rice plant (pHY105, Ubi::OsDhar; pHY102, SWPA2::BrMdhar).

FIG. 2 illustrates RT-PCR analysis of transgenic rice plant in which OsDHAR gene or BrMDHAR is overexpressed under high salinity.

FIG. 3 illustrates salt stress resistance analysis of a transgenic rice plant.

FIG. 4 illustrates phenotype analysis of a transgenic rice plant in a GMO test field.

FIG. 5 illustrates enzyme activity analysis of a transgenic rice plant under salt stress.

FIG. 6 illustrates low temperature stress resistance analysis of a transgenic rice plant.

FIG. 7 illustrates growth rate of a transgenic rice plant after seeding and planting.

FIG. 8 illustrates the tiller number that is directly related to the crop yield of a transgenic rice plant.

FIG. 9 illustrates enzyme activity analysis of a transgenic rice plant under stress conditions.

FIG. 10 illustrates the measurement of growth length of T₃ transgenic rice plant under low temperature stress conditions.

FIG. 11 illustrates salt stress resistance analysis of a transgenic rice plant.

FIG. 12 illustrates growth rate of a transgenic rice plant from planting on June, 2010 to harvest on October.

FIG. 13 illustrates the analysis relating to total antioxidant activity and lipid oxidation of a transgenic rice plant.

FIG. 14 illustrates a property determination for agronomic traits of T₃ and T₄ transgenic rice plant (TPW, total plant weight; CW, culm weight; RW, root weight; NP, number of panicles per hill; NSP, number of spikelets per panicle; FR, filling rate; TGW, total grain weight; 1000 GW, 1000 grain weight).

FIG. 15 illustrates determination of sensitivity of rice plant mutant with T-DNA insertion against salt stress.

FIG. 16 illustrates molecular biological functional analysis of rice plant mutant with T-DNA insertion (A: #1-4 (OsMdhar) and #6-3 (OsDhar) as a rice plant mutant with T-DNA insertion were subjected to semi RT-PCR analysis for determining expression of genes related to anti-oxidation, B; MDHAR enzyme activity analysis of #1-4 (OsMdhar) as a rice plant mutant with T-DNA insertion).

FIG. 17 illustrates morphology analysis of a rice plant mutant with T-DNA insertion in a test field (A: number of effective tiller of a rice plant mutant after seeding).

FIG. 18 illustrates growth rate of a rice plant mutant with T-DNA insertion from seeding on May, 2010 to harvest.

FIG. 19 illustrates a property determination for agronomic traits of a rice plant mutant with T-DNA insertion in test field (TPW, total plant weight; CW, culm weight; RW, root weight; NP, number of panicles per hill; NSP, number of spikelets per panicle; FR, filling rate; TGW, total grain weight; 1000 GW, 1000 grain weight).

### DETAILED DESCRIPTION

To achieve the object of the present invention described above, the present invention provides a method for increasing plant crop yield comprising transforming a plant cell with a recombinant vector containing dhar (dehydroascorbate reductase) gene derived from rice (Oryza sativa) or mdhar (monodehydroascorbate reductase) derived from Chinese cabbage (Brassica rapa) and overexpressing the dhar gene or mdhar gene.

Each of dhar gene and mdhar gene may be present as a multigene family, and each may preferably consist of the nucleotide sequence of SEQ ID NO: 1 or SEQ ID NO: 2, but not limited thereto. Further, the variants of the aforementioned nucleotide sequence are also included in the scope of the present invention. Specifically, the above described gene may comprise a nucleotide sequence which has preferably at least 70%, more preferably at least 80%, still more preferably at least 90%, and most preferably at least 95% homology with the nucleotide sequence of SEQ ID NO: 1 or SEQ ID NO: 2. The “sequence homology %” for a certain polynucleotide is identified by comparing a comparative region with two sequences that are optimally aligned. In this regard, a part of the polynucleotide in comparative region may comprise an addition, replacement or deletion(i.e., a gap) compared to a reference sequence (without any addition or deletion) relative to the optimized alignment of the two sequences.

According to the method of the present invention, the increased plant crop yield can be increased tiller number of a plant, but not limited thereto.

The term “recombinant” indicates a cell which replicates a heterogeneous nucleotide or expresses said nucleotide, or a peptide, a heterogeneous peptide, or a protein encoded by a heterogeneous nucleotide. Recombinant cell can express a gene or a gene fragment in a form of a sense or antisense, that is not found in natural state of cell. In addition, a recombinant cell can express a gene that is found in natural state, provided that said gene is modified and re-introduced into the cell by an artificial means.

According to the present invention, the sequence of dhar gene or mdhar gene can be incorporated to the recombinant expression vector. The term “recombinant expression vector” means bacteria plasmid, phage, yeast plasmid, plant cell virus, mammalian cell virus, or other vector. Any plasmid and vector can be generally used if it can replicate and is stabilized in a host. Important characteristics of the expression vector include that it comprises a replication origin, a promoter, a marker gene, and a translation control element.

The expression vector comprising dhar gene or mdhar gene sequence and a suitable transcription/translation control element can be constructed according to a method which is well known to a skilled person in the art. The method includes an in vitro recombinant DNA technique, a DNA synthesis technique, and an in vivo recombinant technique. For inducing mRNA synthesis, the DNA sequence can be effectively linked to a suitable promoter present in the expression vector. In addition, the expression vector may comprise a ribosome binding site as a translation initiation site and a transcription terminator.

Preferred example of the recombinant vector of the present invention is Ti-plasmid vector which can transfer a part of itself, i.e., so called T-region, to a plant cell when the vector is present in an appropriate host such as Agrobacterium tumefaciens. Other types of Ti-plasmid vector (see, EP 0 116 718 B1) are currently used for transferring a hybrid DNA sequence to protoplasts that can produce a new plant by appropriately inserting a plant cell or hybrid DNA to a genome of a plant. Especially preferred form of Ti-plasmid vector is a so-called binary vector which has been disclosed in EP 0 120 516 B1 and U.S. Pat. No. 4,940,838. Other vector that can be used for introducing the DNA of the present invention to a host plant can be selected from a double-stranded plant virus (e.g., CaMV), a single-stranded virus, and a viral vector which can be originated from Gemini virus, etc., for example a non-complete plant viral vector. Use of said vector can be advantageous especially when a host plant cannot be easily transformed.

Expression vector may comprise at least one selective marker. Said selective marker is a nucleotide sequence having a property of being selected by a common chemical method. Examples include all genes that are useful for distinguishing transformed cells from non-transformed cells. Specific examples thereof include a gene resistant to herbicide such as glyphosate and phosphinotricine, a gene resistant to antibiotics such as kanamycin, G418, bleomycin, hygromycin, and chloramphenicol, and aadA gene, but not limited thereto.

For the recombinant vector according to the present invention, a promoter can be any of CaMV 35S, actin, ubiquitin, pEMU, MAS, histone promoter, SWPA2 promoter, and Clp promoter, but not limited thereto. The term “promoter” means a DNA molecule to which RNA polymerase binds in order to initiate its transcription, and it corresponds to a DNA region upstream of a structural gene. The term “plant promoter” indicates a promoter which can initiate transcription in a plant cell. The term “constitutive promoter” indicates a promoter which is active in most of environmental conditions and development states or cell differentiation states. Since a transformant can be selected with various mechanisms at various stages, the constitutive promoter can be preferable for the present invention. Therefore, a possibility for choosing the constitutive promoter is not limited herein.

For the recombinant vector of the present invention, any conventional terminator can be used. Examples include nopaline synthase (NOS), rice α-amylase RAmy1 A terminator, phaseoline terminator, a terminator for optopine gene of Agrobacterium tumefaciens, and rnnB1/B2 terminator of Escherichia coli, but are not limited thereto. Regarding the necessity of terminator, it is generally known that such region can increase reliability and an efficiency of transcription in plant cells. Therefore, the use of terminator is highly preferable in view of the contexts of the present invention.

With respect to a host cell having an ability of having stable and continuous cloning and expression of the vector of the present invention in prokaryotic cells, any one known in the pertinent art can be used. Examples thereof include, Bacillus sp. strain including E. coli JM109, E. coli BL21, E. coli RR1, E. coli LE392, E. coli B, E. coli X 1776, E. coli W3110, Bacillus subtillus, Bacillus thuringiensis and the like, and intestinal bacteria and strains including Salmonella typhimurium, Serratia marcescens and various Pseudomonas sp. etc.

In addition, when an eukaryotic cell is transformed with the vector of the present invention, yeast (Saccharomyces cerevisiae), an insect cell, a human cell (for example, CHO (Chinese hamster ovary) cell line, W138, BHK, COS-7, 293, HepG2, 3T3, RIN and MDCK cell line), a plant cell, and the like can be used as a host cell. The host cell is preferably a plant cell.

When a host cell is a prokaryotic cell, delivery of the vector of the present invention into a host cell can be carried out by CaCl₂ method, Hanahan's method (Hanahan, D., J. Mol. Biol., 166:557-580 (1983)) or an electroporation method, and the like. In addition, when a host cell is an eukaryotic cell, the vector can be introduced to a host cell by a microinjection method, calcium phosphate precipitation method, an electroporation method, a liposome-mediated transfection method, DEAE-dextran treatment method, or a gene bombardment method, and the like.

The present invention further provides a method for producing a plant with increased crop yield comprising transforming a plant cell with a recombinant vector containing dhar (dehydroascorbate reductase) gene derived from rice (Oryza sativa) or mdhar (monodehydroascorbate reductase) derived from Chinese cabbage (Brassica rapa) and regenerating the plant from the transformed plant cell. Preferably, the dhar gene or mdhar gene may consist of the nucleotide sequence of SEQ ID NO: 1 and SEQ ID NO: 2, respectively.

According to the plant production method of the present invention, the increased plant crop yield may be an increased tiller number of a plant, but not limited thereto.

The method of the present invention comprises transforming a plant cell with the recombinant vector of the present invention, and the transformation may be mediated by Agrobacterium tumefiaciens. Further, the method of the present invention comprises a regenerating a transgenic plant from the transformed plant cell. As for the method for regenerating a transgenic plant from a transformed plant cell, a method well known in the pertinent art can be used.

The transformed plant cell needs to be regenerated into a whole plant. Techniques for regeneration into a mature plant by culture of callus or protoplast are well known in the pertinent art for various species (Handbook of Plant Cell Culture, Vol. 1-5, 1983-1989 Momillan, N.Y.).

The present invention also provides a transgenic plant with increased crop yield that is produced by the aforementioned method, a seed thereof. The plant can be preferably a monocot plant, but not limited thereto. Preferred examples thereof include Alismataceae, Hydrocharitaceae, Juncaginaceae, Scheuchzeriaceae, Potamogetonaceae, Najadaceae, Zosteraceae, Liliaceae, Haemodoraceae, Agavaceae, Amaryllidaceae, Dioscoreaceae, Pontederiaceae, Iridaceae, Burmanniaceae, Juncaceae, Commelinaceae, Eriocaulaceae, Graminease (Poaceae), Araceae, Lemnaceae, Sparganiaceae, Typhaceae, Cyperaceae, Musaceae, Zingiberaceae, Cannaceae, and Orchidaceae, but not limited thereto.

The present invention also provides a composition for increasing crop yield of a plant containing dhar (dehydroascorbate reductase) gene derived from rice (Oryza sativa) or mdhar (monodehydroascorbate reductase) derived from Chinese cabbage (Brassica rapa). Preferably, the dhar gene or mdhar gene may consist of the nucleotide sequence of SEQ ID NO: 1 and SEQ ID NO: 2, respectively. The composition comprises rice-derived dhar gene or Chinese cabbage-derived mdhar gene as an effective component, and by transforming a plant with the gene, plant crop yield can be increased.

The present invention also provides a method for enhancing resistance of a plant to environmental stress comprising transforming a plant cell with the recombinant vector containing dhar (dehydroascorbate reductase) gene derived from rice (Oryza sativa) or mdhar (monodehydroascorbate reductase) derived from Chinese cabbage (Brassica rapa) and overexpressing dhar gene or mdhar gene. Preferably, the dhar gene or mdhar gene may consist of the nucleotide sequence of SEQ ID NO: 1 and SEQ ID NO: 2, respectively.

With regard to the method of the present invention, the environmental stress may be salt, low temperature, or oxidative stress, but not limited thereto.

The present invention also provides a method for producing a plant with increases resistance to environmental stress comprising transforming a plant cell with the recombinant vector containing dhar (dehydroascorbate reductase) gene derived from rice (Oryza sativa) or mdhar (monodehydroascorbate reductase) derived from Chinese cabbage (Brassica rapa) and regenerating a plant from a transformed plant cell. Preferably, the dhar gene or mdhar gene may consist of the nucleotide sequence of SEQ ID NO: 1 and SEQ ID NO: 2, respectively.

With regard to the method for producing a plant of the present invention, the environmental stress may be salt, low temperature, or oxidative stress, but not limited thereto.

The present invention also provides a transgenic plant with enhanced resistance of a plant to environmental stress that is produced by the aforementioned method, and a seed thereof. The plant is preferably a monocot plant, but not limited thereto.

The present invention also provides a composition for enhancing resistance to environmental stress containing dhar (dehydroascorbate reductase) gene derived from rice (Oryza sativa) or mdhar (monodehydroascorbate reductase) derived from Chinese cabbage (Brassica rapa). Preferably, the dhar gene or mdhar gene may consist of the nucleotide sequence of SEQ ID NO: 1 and SEQ ID NO: 2, respectively. The composition comprises rice-derived dhar gene or Chinese cabbage-derived mdhar gene as an effective component, and by transforming a plant with each gene, resistance of a plant to environmental stress can be enhanced.

Herein below, the present invention is explained in greater detail in view of the Examples. However, it is evident that the following Examples are given only for exemplification of the present invention and by no means the present invention is limited to the following Examples.

1. Materials and Methods

(1) Construction of Vector for Overexpressing Genes of Monodehydroascorabate Reductase (MDHAR) and Dehydroascorbate Reductase (DHAR) and Development of T₁ Generation Rice Plant Transformed with Vector and Seed Thereof.

Full length cDNA encoding dehydroascorbate reductase (DHAR) (AY074786/Os05g0116100) was obtained by reverse transcription polymerase chain reaction (RT-PCR) method from total RNA of Oryza sativa cv. Ilmi, and it was named OsDHAR1. It was then cloned between the corn ubiquitin promoter of pGA1611 and nos terminator. The fragment of 3.6 kb containing ubiquitin promoter:: OsDHAR1::nos terminator was cloned into pCAMBIA3300 to prepare pOsDHAR1. pOsDHAR1 binary vector was used for transduction of Agrobacterium strain LBA4404. Transformation and selection of a plant, regeneration of bar-resistant callus were performed by the method described above (Kang et al., 1998 Plant Mol Biol 38:1021-1029). In order to prepare other construct containing OsDHAR gene, OsDHAR gene digested with NcoI and KpnI was inserted into a corresponding site in pCAMBIA3300 plant insertion vector containing SWPA2 promoter, nos terminator, and bar gene as a selection marker.

As a host for overexpression of pOsDHAR1, Ilmi variant of Oryza sativa L. japonica was used. T-DNA insertion mutant (3A-03259) in which any of dehydroascorbate reductase gene (OsDHAR2, Os06g0232600) has been knocked down was Dongjin variant. 3A-03259 can be found in the website of RiceGE. For observing genotype and phenotype, the transgenic rice plant was cultivated with a non-transgenic rice plant from June to October in GMO field (Gunwi campus; 36° 24′ N, 128° 53′ E and 95 m a.s.1.) of Kyungpook National University (Daegu, South Korea). The rice plant at T₃ generation was used for determining resistance to a abiotic stress such as salt, low temperature, H₂O₂, drought, and PEG (polyethylene glycol).

The rice plant transformed with ubiquitin promoter:: OsDHAR1 was identified by PCR which uses Ubi-F (5′-tgccttcatacgctatttatttgcttg-3′: SEQ ID NO: 3) and OsDHAR-R2 (5′-ccttgctcttcaagaacgttgtgaagc-3′: SEQ ID NO: 4) primers. 3A-03259 was identified with gene-specific primer RP (5′-ccgttaataaatggaccctgc-3′: SEQ ID NO: 5) and LP (5′-aagcgcaattttacagctgag-3′: SEQ ID NO: 6).

cDNA encoding Chinese cytoplasmic MDHAR (NCBI accession number: AY039786) was obtained by RT-PCR method from total RNA of Brassica rapa var. pekinensis. It was then inserted to TOPO-TA vector (Invitrogen) and identified by sequencing. In order to prepare a construct containing BrMDHAR gene, BrMDHAR gene digested with NcoI and KpnI was inserted into a corresponding site in pCAMBIA3300 plant insertion vector containing SWPA2 promoter, nos terminator, and bar gene as a selection marker. The resulting construct was used for transduction of Agrobacterium strain LBA4404 by electroporation. Transformation selection of Oryza sativa L. japonica (Ilmi), and regeneration of bar-resistant callus were performed by the method described above (Kang et al., 1998 Plant Mol Biol 38:1021-1029). In order to prepare other construct containing BrMDHAR gene, BrMDHAR gene digested with HindIII and KpnI was inserted into a corresponding site in pGA1611 plant insertion vector containing Ubi promoter, nos terminator, and hygromycin resistance gene as a selection marker. The resulting construct (pGA1611::BrMDHAR) was inserted to pCAMBIA3300 containing nos terminator and bar gene as a selection marker by blunt end ligation method using BamHI and SacII for insertion of a construct (Ubi::BrMDHAR).

Fifty independent T₀ transgenic rice plants (TP) which express BrMDHAR were grown until mature phase in the natural paddy field conditions. T₁ seeds were collected and T₁ offsprings obtained from Tₒ rice plant were subjected to a segregation pattern analysis (Mendelian law) using SWPA2-F (5′-caatcaagcattctacttctattgcagc-3′: SEQ ID NO: 7) and BrMDHAR-R (5′-caatctcagaacagtagagccagttgc-3′: SEQ ID NO: 8) primers. About 1000 T₂ seeds (20 seeds per line) harvested from T₁ rice plant were germinated in a rooting medium containing herbicide. Screening regarding germination of T₂ seeds, which have been induced from T₁ rice plant, in the rooting medium containing herbicide was repeatedly performed until all of the T₂ seeds harvested from geminated T₁ plant are analyzed. In order to determine a homozygous plant, PCR analysis, Western blot, and enzyme activity analysis were performed.

(2) Determination of Resistance to Environmental Stress in Seed of T₂ Rice Plant Having Overexpressed MDHAR and DHAR Gene

After selecting two homolines of pHY101 (Ubi::BrMdhar) transformant, and one homoline of pHY102 (SWPA2::BrMdhar), and two homolines of pHY104 (SWPA2::OsMdhar) transformant in February, 2008 followed by seeding, genotype and resistance to stress (salt and low temperature) were examined.

After selecting two homolines of pHY101 transformant, one homoline of pHY102, and two homolines of pHY104 transformant in June, 2008, twelve seedlings for each line of them were grown in a GMO test field for transgenic plant of Kyungpook National University located Hyoryung-myun, Gunwi-gun, Gyeongsangbuk-do, South Korea. Then, their characters were observed and a biological analysis was made.

In December, 2008, T₂ seeds of two homolines of T₂ generation pHY101 transformant, one homoline of pHY102, and two homolines of pHY104 transformant were planted in a greenhouse and grown for three weeks. The grown plant was then subjected to an analysis regarding RNA expression, enzyme activity, and microarray under stress conditions (salt, low temperature, and oxidation).

After the treatment with salt stress (0.1 M) for six days with seeding grown in a greenhouse, leaves were collected and soaked in liquid N₂. The proteins were extracted and analyzed in terms of enzyme activity. The protein extraction was performed by using an extraction buffer containing 50 mM Tris-HCl (pH 7.5), 1 mM EDTA, 1 mM MgCl₂, 1 mM PMSF, and protease inhibitor cocktails. RNA was extracted from leaf tissue of pHY101, pHY102, pHY104, and a control Ilmi rice plants by using RNeasy Plant Mini Kit (QIAGEN, USA).

(3) Determination of Resistance of T₂, T₃ and T₄ Rice Plant with Overexpressed MDHAR and DHAR Gene and Observation of Phenotypes

\textcircled{1} Analysis of Resistance to Stress, Biological Analysis, and Transgene Stability Analysis After Development of Seeds of T₂ Generation

After selecting two homolines of pHY103 (Ubi::OsMDHAR) transformant, seven homolines of pHY105 (Ubi::OsDHAR), and seven homolines of pHY106 (SWPA2::OsDHAR) transformant in February and June, 2009 followed by seeding in a greenhouse, resistance to stress (salt and low temperature) was examined.

After selecting two homolines of pHY103 transformant, seven homolines of pHY105, and seven homolines of pHY106 transformant in June, 2009, twelve seedlings for each line of them were grown in a GMO test field for transgenic plant of Kyungpook National University located Hyoryung-myun, Gunwi-gun, Gyeongsangbuk-do, South Korea. Then, their characters were observed and a biological analysis was made.

In order to determine the segregation ratio of two homolines of pHY103 transformant, seven homolines of pHY105, and seven homolines of pHY106 transformant which have been planted in a greenhouse in February, 2009, genotypes were analyzed.

Fifteen T₂ seeds were developed in a GMO paddy field, and then genotype of the development line was determined by using PCR with DNA isolated from leaf tissue. PCR conditions were as follows: 95° C., 5 min; 94° C., 1 min; 57° C., 1 min; 72° C., 1 min (35 cycles); and 72° C., 7 min.

\textcircled{2} Analysis of Resistance to Stress, Biological Analysis, and Transgene Stability Analysis after Development of Seeds of T₃ Generation

pHY101 transformant, pHY102 transformant, and pHY104 transformant were sown in a greenhouse in February and June, 2009, and resistance to stress (salt and low temperature) was examined

Phenotype of the 4 week-old transgenic rice plant sown in the greenhouse was observed. Observation result of the phenotype after treatment with 100 mM salt and treatment with low temperature of 10° C., which have been performed after sowing in the greenhouse, was analyzed in terms of the salt or low temperature treatment period and compared to the control group, i.e., Ilmi rice.

In June, 2009, twelve seedlings for each of pHY101 transformant, pHY102 transformant, and pHY104 transformant were grown in a GMO test field for transgenic plant of Kyungpook National University located Hyoryung-myun, Gunwi-gun, Gyeongsangbuk-do, South Korea. Then, their characters were observed and a biological analysis was made.

pHY101 transformant, pHY102 transformant, and pHY104 transformant which have been sown in a green house in February and June, 2009 were analyzed for expression of RNA and protein, and enzyme activity with regard to adaptation to salt and low temperature.

RNA was isolated by using Tri-reagent solution (Molecular Research Center, INC.), and cDNA synthesis was performed by RT-PCR using MMLV reverse transcriptase and oligo dT₁₈ primer (Promega).

After the treatment with salt stress for six days after seeding in a greenhouse, leaves were collected and the proteins were extracted and analyzed in terms of enzyme activity. The protein extraction was performed by using an extraction buffer containing 50 mM Tris-HCl (pH 7.5), 1 mM EDTA, 1 mM MgCl₂, 1 mM PMSF, protease inhibitor cocktails.

Measurement of MDHAR activity was performed by following a change in absorbance at 340 nm (e=6.2 mM⁻¹ cm⁻¹) in 50 mM phosphate buffer (pH 7.2) solution containing 0.2 mM NADH, 2 mM ascorbate (AsA), and 1 unit of ascorbate oxidase, wherein the absorbance reflects the amount of AsA reduced by monodehydroascorbate (MDHA). Protein concentration was measured by Bradford's method using Protein Dye Reagent (Bio-Rad, Hercules, USA).

\textcircled{3} Determination of Phenotypes of T₂, T₃ and T₄ Generation Rice Plant in a GMO Test Field

Phenotypes of the transgenic rice plant obtained by sowing in the test field in May, 2010 were observed. Phenotype observation after planting in the test field was to analyze the number of effective tiller, total fresh weight, and harvest amount from vegetative growth to reproductive growth compared to the control group, i.e., Ilmi rice.

(4) Determination of Expression of OsMdhar and OsDhar Gene in Rice Plant Mutant with T-DNA Insertion

Seeds of an insertional rice plant mutant with knock-down OsMdhar gene and knock-out OsDhar gene were obtained from POSTECH and developed in a test field to determine the genotype of T-DNA insertion. The phenotype was also observed.

(5) Analysis of Sensitivity of Mutant Rice Plant with T-DNA Insert to Stress Under Specific Oxidative Condition

A mutant with T-DNA insertion was developed in a greenhouse. After growing the seedlings at 30° C. for 2 weeks, genomic DNA was isolated from the seedlings and genotyping was performed by using PCR for determining the homozygous plants of development line. For isolation of genomic DNA, a grind buffer was prepared by mixing at ratio of 4:1, a homogenizing buffer (0.1 M NaCl, 0.2 M sucrose, 0.01 M EDTA, 0.03 M Tris-HCl, pH 8.0) and dissolution buffer (0.25 M EDTA, 2.5% (w/v) SDS, 0.5 M Tris-HCl, pH 9.2) was used. Plant tissues (0.2 g) were frozen in liquid nitrogen and ground in a 1.5 ml tube. After that, it was added with 400 μl grind buffer, kept at 70° C. for 30 minutes, and then subjected to centrifuge (12000 rpm, 10 min). The supernatant was added with 70% ethanol (final concentration) and then the supernatant was removed after centrifugation (12000 rpm, 10 min). After washing cell pellets twice with 70% ethanol, the precipitates were air-dried, and dissolved in distilled water at 50° C. PCR conditions were as follows: 93° C., 3 min; 93° C., 1 min; 54° C., 1 min; 72° C., 1 min (35 cycles); and 72° C., 7 min.

Two lines of OsMdhar knock-down and four lines of OsDhar knock-out, which have been sown in a greenhouse in February, 2008, were subjected to analysis for RNA expression and enzyme activity.

After sowing the mutant with T-DNA insertion in a greenhouse followed by growing for about three weeks at 30° C. or so, the obtained seedlings were treated with salt and low temperature stress. A sample was taken after approximately six days following stressor, and after separating proteins, protein expression and enzyme activity were determined Plant tissues (0.2 g) were ground by using a mortar and a pestle. The ground tissues were added in portions to a microcentrifuge tube by using a baked spatula or all of them were added to a 15 ml conical tube. Protein extraction from the plant was performed by adding a homogenizing buffer (50 mM Tris-HCl (pH8.0), 150 mM NaCl, 1 mM EDTA, 1% NP-40, 1 mM PMSF, protease inhibitor cocktails). After adding the homogenizing buffer, vortexing was performed. After keeping it on ice for 10 minutes, it was centrifuged for 20 minutes at 12,000 rpm at 4° C. The cleared supernatant was collected and transferred to a new tube. The extracted protein was used for measuring protein expression and enzyme activity.

The homozygote lines including one of OsMdhar knock-down series and one of OsDhar knock-out series, which have been sown in a greenhouse in February and June, 2009, were analyzed in terms of stress sensitivity to salt and cold.

Expression of RNA and protein, and enzyme activity analysis was performed for the plants of OsMdhar knock-down and OsDhar knock-out, which have been sown in a greenhouse in February and June, 2009.

In June, 2009, twelve lines for each of OsMdhar knock-down and OsDhar knock-out were developed in a GMO test field for transgenic plant of Kyungpook National University located Hyoryung-myun, Gunwi-gun, Gyeongsangbuk-do, South Korea. Then, analysis of their characters was made.

Phenotypes of the transgenic rice plant obtained by planting in the GMO test field in June, 2010 were observed. Phenotype observation after sowing in the same field was to analyze the number of effective tiller, total fresh weight, and harvest amount in early growth phase (vegetative stage) and late growth phase (reproductive stage) compared to the control group, Dongjin rice.

### Example 1

### Determination of Salt Resistance of T₂ Transgenic Rice Plant Derived from Seed of T₁ Transgenic Rice Plant with Overexpressed MDHAR and DHAR Gene

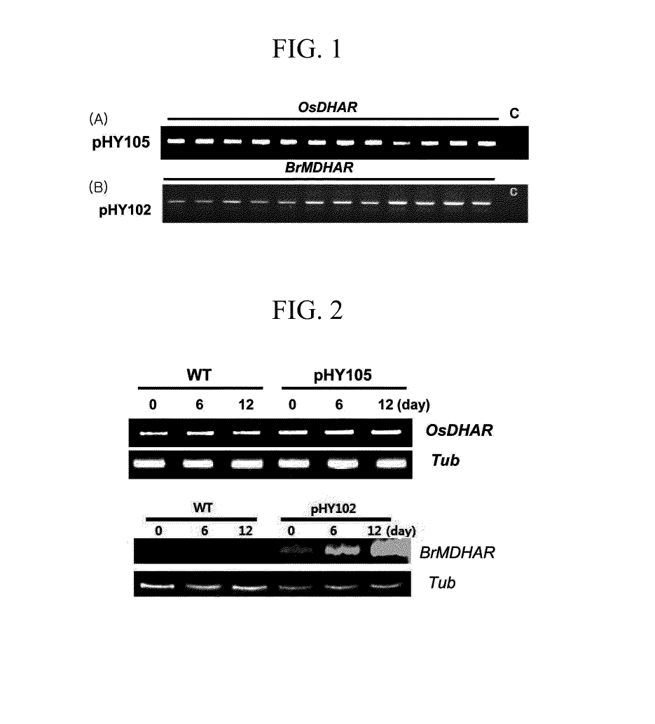

In order to confirm the genes inserted to the plant, genotyping was tried using PCR after extracting genomic DNA from pHY101, pHY102, pHY104 and pHY105, which has been obtained by sowing seeds of T₁ transgenic rice plant overexpressing MDHAR and DHAR gene in February, 2008. As a result, it was found that there is a correlation between the plant having resistance to salt stress and inserted gene (FIG. 1). DNA was extracted from plants of pHY105 and pHY102 and genotype was analyzed. As a result, it was confirmed that there are an inserted gene in every plant of pHY105 line and pHY102 line except the control group (Ilmi rice).

In order to determine increased expression of the inserted gene in transgenic rice plant, RNA was extracted from leaf tissue of pHY101, pHY102, pHY104 and pHY105 plants sown in a greenhouse in February, 2008, and an expression analysis was performed by semi-quantitative RT-PCR. As a result, it was found that the transgenic rice plant having resistance to oxidative stress has a high expression level of mRNA coding the insert gene. In the transgenic plant grown without a treatment of salt stress, expression amount of RNA of the insert gene was low, and the RNA expression amount was shown to gradually increase in accordance with salt stress treatment period. Based on the results, it was confirmed that the transgenic rice plant has resistance to salt stress (FIG. 2). It was also confirmed that, compared to WT (Ilmi rice), the expression level of the antioxidative-related genes increases more in the transgenic plant in accordance with salt stress treatment period.

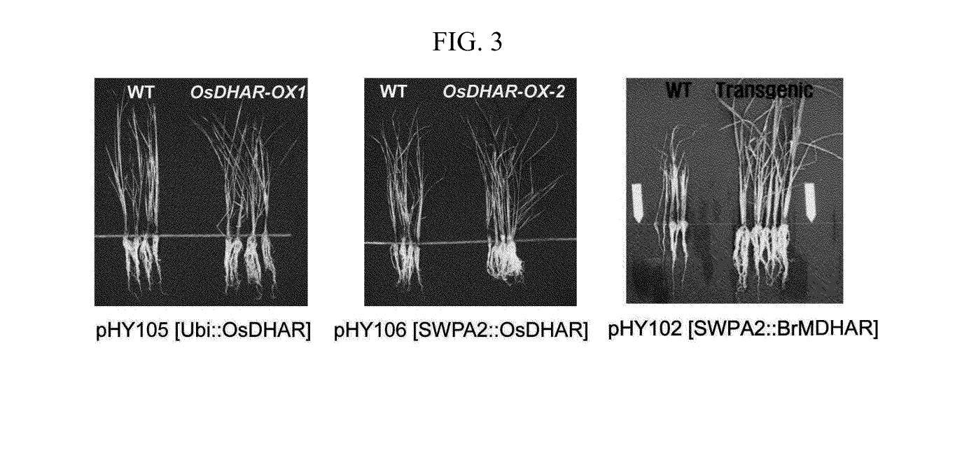

Phenotype of the transgenic rice plant sown in a greenhouse and Gunwi GMO field was determined. According to the phenotype observation after sowing in a greenhouse and treatment with salt stress, it was found that the transgenic rice plant has higher resistance to stress in accordance with salt stress treatment period than the control group, i.e., Ilmi rice. As for the salt stress treatment period, the plants were grown for 15 days after sowing the seeds, and the salt stress treatment was performed for about 40 days at 100 mM NaCl (FIG. 3). The transgenic rice plant pHY105, pHY106 and pHY102 showed higher resistance to salt stress than WT (Ilmi rice). Salt stress with 100 mM NaCl was applied for 40 days and recovered for 10 days under NaCl-free water.

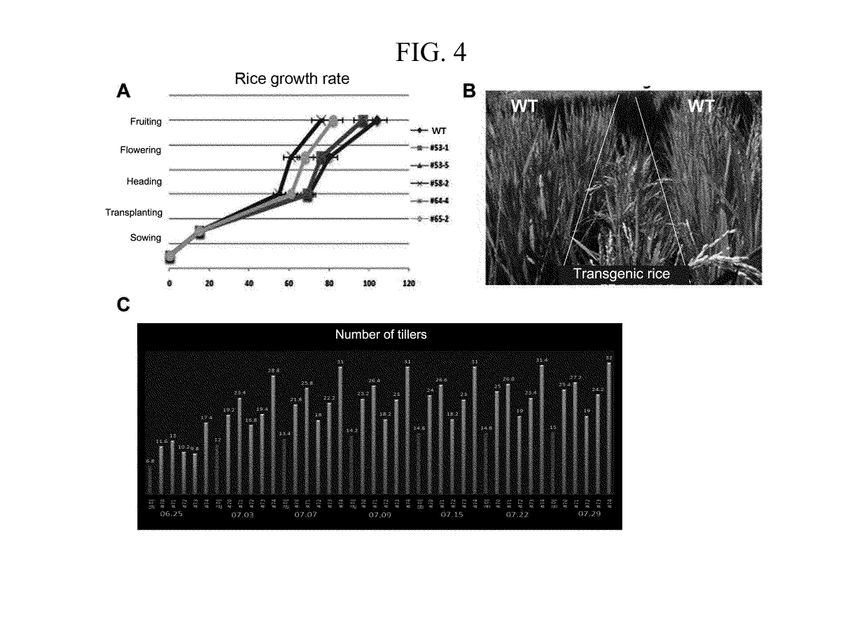

For phenotype observation after sowing in Gunwi, development in a GMO test field for transgenic plant of Kyungpook National University located Hyoryung-myun, Gunwi-gun, Gyeongsangbuk-do, South Korea was performed in June, 2008, and the agronomic traits were continuously observed from the sowing. As a result, it was confirmed that the transgenic plants have better early growth phase and late growth phase than the control group (Ilmi rice). Based on the result, it was found that the transgenic plant has better growth rate compared to Ilmi rice even in a common paddy field condition (FIG. 4). Growth rate of the transgenic rice plant was determined from the sowing. According to determination of early growth phase and late growth phase of the control group (Ilmi rice), #53-1 (pHY101), #53-5 (pHY101), #58-2 (pHY102), #64-4 (pHY104) and #65-2 (pHY104), the transgenic rice plant showed faster growing than the control group, i.e., Ilmi rice (FIG. 4A), and the heading period was 10 to 20 days faster in the transgenic rice plant than the control group, i.e., Ilmi rice (FIG. 4B). FIG. 4C shows the result of determining tiller number, which is in direct relationship with productivity of the transgenic plant. The tiller number in the transgenic plant has increased compared to the control group (Red, control group; Green, transgenic plant).

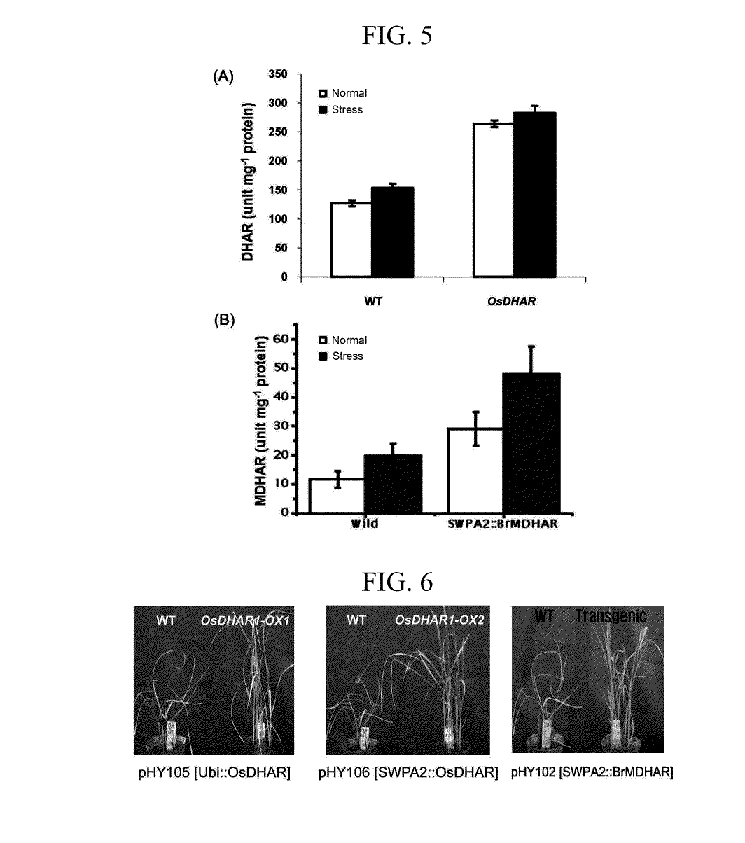

After salt stress treatment with seedling plants grown in a greenhouse, the leaves were collected, and crude protein extract was isolated for measuring enzyme activity of DHAR and MDHAR. As a result of analyzing DHAR or MDHAR enzyme activity, it was confirmed that the transgenic plant exhibited higher enzyme activity in accordance with salt stress treatment period compared to the control group, i.e., Ilmi rice. Based on the results, it was found that, in a general state, the transgenic plant exhibits higher enzyme activity than the control group, Ilmi rice, and under the salt stress condition, the enzyme activity has further increased. Thus, it was confirmed that the transgenic plant has better resistance to salt stress condition than the control group, Ilmi rice (FIG. 5).

According to the enzyme activity analysis for the control group, Ilmi rice, and pHY105 (Ubi::OsDHAR) and pHY102 (SWPA2::BrMDHAR) as a transgenic rice plant, it was confirmed that the transgenic rice plant exhibits higher enzyme activity under salt stress than the control group, Ilmi rice. The results are obtained by using the leaves after treatment for 12 days with 100 mM NaCl(FIG. 5A and FIG. 5B).

### Example 2

### Determination of Resistance to Cold Temperature Stress (Cold Resistance) of T₂ and T₃ Transgenic Rice Plant with Overexpressed MDHAR and DHAR Gene

Phenotype of the transgenic rice plant sown in a greenhouse was observed. According to the phenotype observation after low temperature stress treatment following the sowing in a greenhouse, the transgenic rice plant was found to have higher resistance to low temperature than the control group, Ilmi rice. Transgenic plants was grown for about 30 days after the sowing in a greenhouse, and then, exposed to low temperature (10° C.) for 50 days(FIG. 6).

After the low temperature stress (10° C.). treatment for about 50 days, the plant was recovered for 10 days under normal temperature. As a result, it was found that the transgenic rice plant pHY105, pHY106 and pHY102 have higher resistance to low temperature stress than WT (Ilmi rice).

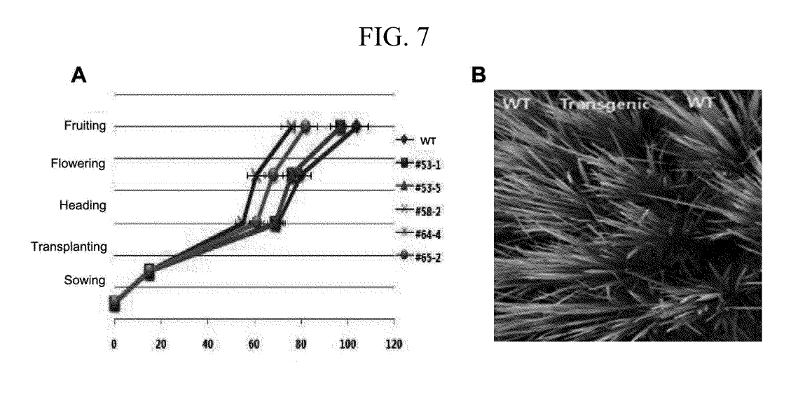

For phenotype observation of T₃ transgenic rice plant after planting in Gunwi, development in a test field for transgenic plant of Kyungpook National University located Hyoryung-myun, Gunwi-gun, Gyeongsangbuk-do, South Korea was performed in June, 2009, and the agronomic characters were continuously observed from the planting. As a result, it was confirmed that the transgenic plants have better early growth phase and late growth phase than the control group (Ilmi rice). Based on the result, it was found that the transgenic rice plant has better growth rate like T₂ transgenic rice plant compared to Ilmi rice even in a common paddy field condition (FIG. 7). According to the determination of early growth phase and later growth phase of the control group (Ilmi rice), #53-1 (pHY101), #53-5 (pHY101), #58-2 (pHY102), #64-4 (pHY104), and #65-2 (pHY104), it was shown that the transgenic plants have better growth than the control group (Ilmi rice) (FIG. 7A). Further, the heading period was 10 to 20 days faster in the transgenic rice plant than the control group, i.e., Ilmi rice (FIG. 7B).

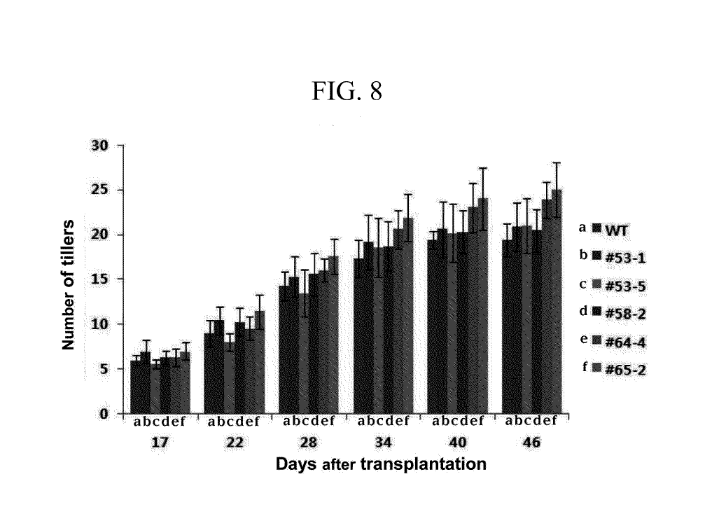

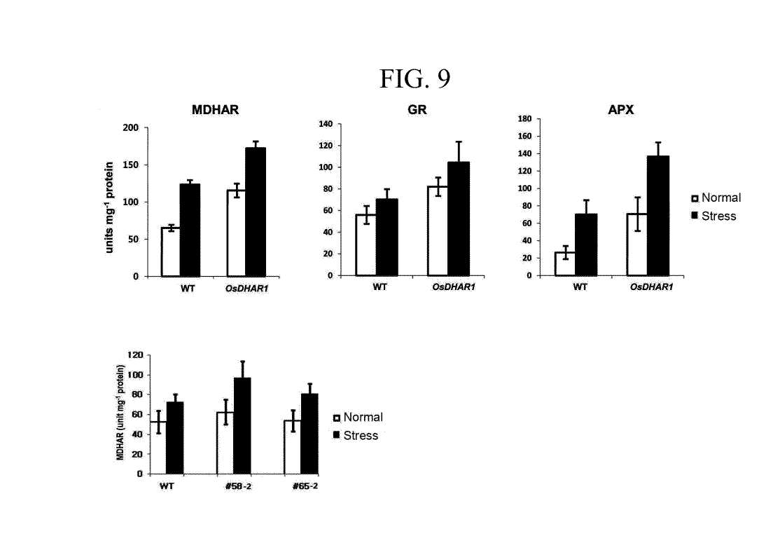

For measurement of the effective tiller number of T₃ transgenic rice plant after planting in Gunwi, development in a GMO test field for transgenic plant of Kyungpook National University located Hyoryung-myun, Gunwi-gun, Gyeongsangbuk-do, South Korea was performed in June, 2009, and the effective tiller number was continuously observed from the planting. As a result, it was confirmed that T₃ transgenic rice plant has better initial tiller number than the control group (Ilmi rice). Based on the result, it was found that the transgenic rice plant has better initial adaptability in a common environmental paddy field than Ilmi rice (FIG. 8). According to the determination of initial tiller number of the control group (Ilmi rice), #53-1 (pHY101), #53-5 (pHY101), #58-2 (pHY102), #64-4 (pHY104), and #65-2 (pHY104), it was found that the transgenic rice plant has better growth state than the control group, i.e., Ilmi rice.

After salt stress treatment followed sowing in a greenhouse, the leaves were collected and the enzyme activity was analyzed. As a result of analyzing the enzyme activity, it was confirmed that the transgenic rice plant exhibited higher enzyme activity compared to the control group, i.e., Ilmi rice, in accordance with the salt stress treatment period. Based on the results, it was found that, in general state, the transgenic rice plant exhibits higher enzyme activity than the control group, Ilmi rice, and under the salt stress, the enzyme activity has further increased. Thus, it was confirmed that the transgenic rice plant has higher resistance to salt stress than the control group, Ilmi rice (FIG. 9). Regarding the low-temperature stress, a sample was collected after growing for 6 days at 10° C. According to the enzyme activity analysis for the control group (Ilmi rice), OsDHAR1 (pHY105), #58-2 (pHY102), and #65-2 (pHY104), it was confirmed that the transgenic rice plant exhibits higher enzyme activity (GR, APX) under low-temperature stress condition than the control group, Ilmi rice.

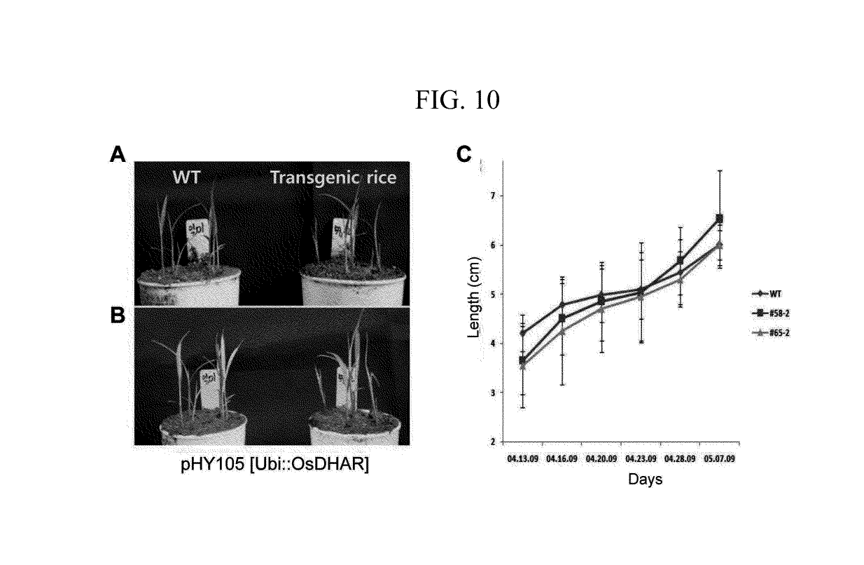

Phenotype of the transgenic rice plant sown in a greenhouse was observed. According to the phenotype observation after low temperature stress treatment following the sowing in a greenhouse, the transgenic rice plant was found to have higher resistance to low temperature than the control group, Ilmi rice, in accordance with the low temperature stress treatment period. The low temperature stress treatment was performed for about 21 days at 10° C. with seedling plants grown for 10 days after sowing in a greenhouse (FIG. 10). Phenotypes of the control variety Ilmi rice and #58-2 (pHY102) were observed. The phenotypes after the low temperatures stress treatment at 10° C. for about 2 weeks were as shown in FIG. 10A and FIG. 10B. As a result of measuring the growth length of Ilmi rice, #58-2 (pHY102), and #65-2 (pHY104) at 10° C., it was found the growth length was higher in the transgenic rice plant than the control variety (FIG. 10C).

### Example 3

### Determination of Antioxidant Activity of T₂ and T₃ Transgenic Rice Plant with Overexpressed MDHAR and DHAR Gene

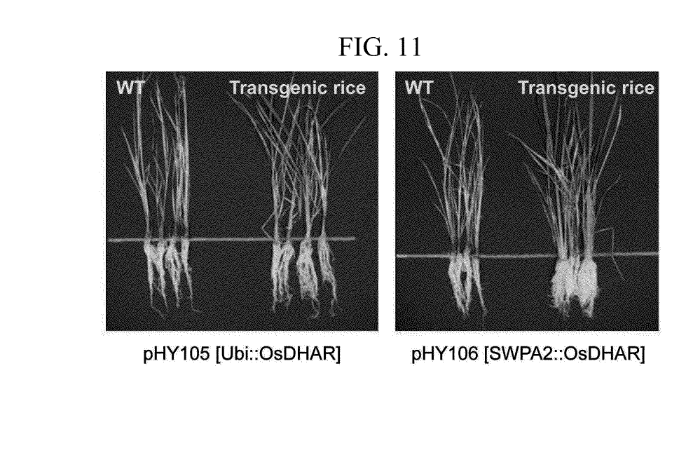

Phenotype of the transgenic rice plant sown in a greenhouse was observed. According to the phenotype observation after salt stress treatment following the sowing in a greenhouse, the transgenic rice plant was found to have higher resistance to salt stress than the control group, Ilmi rice. Seedling plant was grown for about 30 days after the sowing, and it was exposed to 100 mM NaCl for about 40 days. After that, it was recovered for about 14 days under NaCl-free condition (FIG. 11). As a result of the treatment with 100 mM salt stress for about 40 days followed by recovery for 14 days, it was found that the transgenic rice plant pHY105 and pHY106 have higher salt stress resistance than wild-type (WT) plant (Ilmi rice).

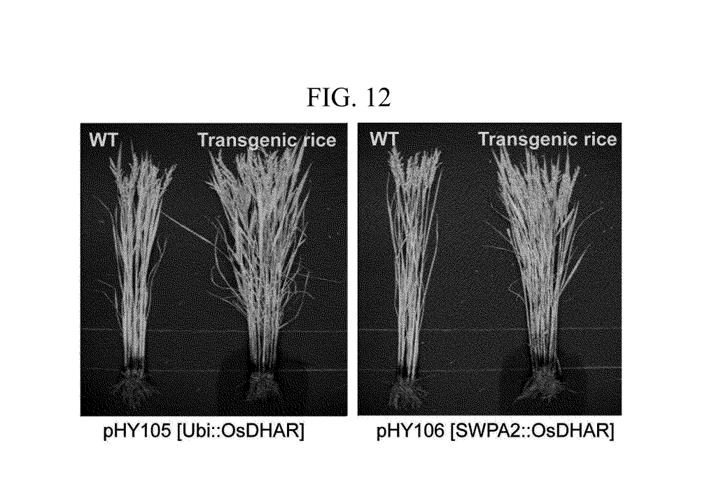

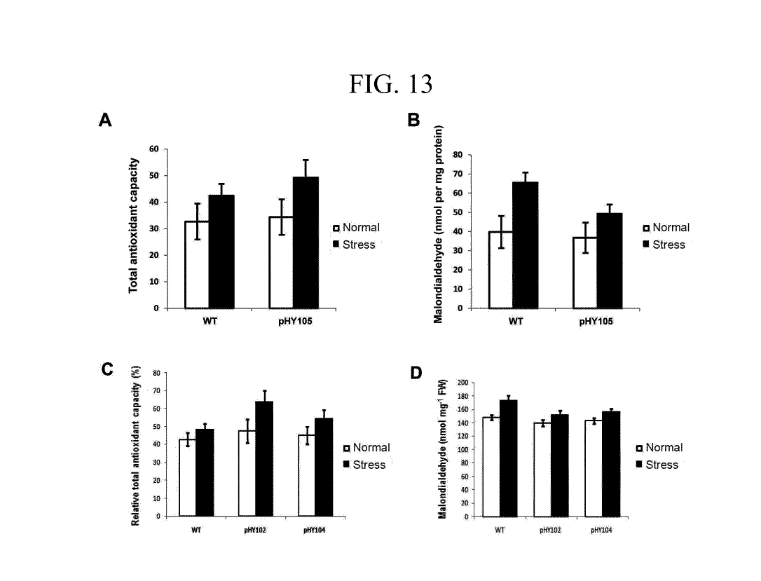

For phenotype observation of T₃ transgenic rice plant after sowing in Gunwi campus, development in a GMO test field for transgenic plant of Kyungpook National University located Hyoryung-myun, Gunwi-gun Gyeongsangbuk-do, South Korea was performed in May, 2010, and the characters were continuously observed from the sowing. As a result, it was confirmed that the transgenic plants have better early growth phase and late growth phase than the control group (Ilmi rice). Based on the result, it was found that the transgenic rice plant has better growth rate like T₂ transgenic rice plant compared to Ilmi rice even in a common natural paddy field condition (FIG. 12). According to the determination of growth state of the control group (Ilmi rice), pHY105, and pHY106, it was shown that both the transgenic rice plants have better growth state than the control group (Ilmi rice).

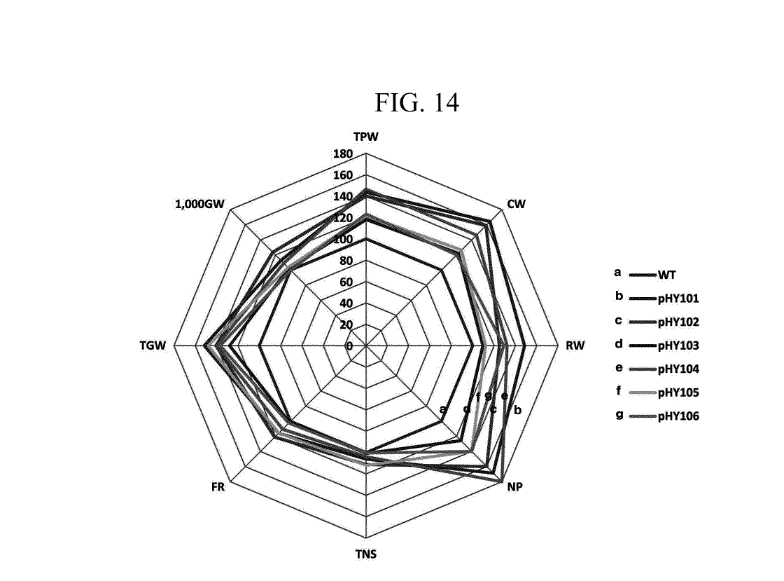

To analyze redox state following salt stress, the leaves were collected, and the proteins were extracted for measuring total antioxidant activity and lipid peroxidation. As a result of analyzing the antioxidant activity, it was confirmed that, in accordance with the period of salt stress treatment, the transgenic rice plant exhibited higher protein expression related to antioxidant system than the control group, i.e., Ilmi rice. Degree of lipid oxidation was analyzed based on analysis of malonaldehyde (MDA) content, which is used as an indicator of lipid oxidation. As a result, in accordance with the period of salt stress treatment, the transgenic rice plant showed lower lipid oxidation than the control group, Ilmi rice. Based on the results, it was confirmed that the transgenic rice plant shows higher antioxidant activity and low lipid oxidation under stress condition than the control group, Ilmi rice. Thus, it was found that the transgenic rice plant has higher resistance to salt stress than the control group, Ilmi rice (FIG. 13). With regard to the salt stress, a sample was collected after growing for 12 days at 100 mM NaCl. According to the antioxidant activity and lipid oxidation analysis of the control group (Ilmi rice), pHY105, pHY102, and pHY104, it was found that the transgenic rice plant has higher antioxidant activity and lower lipid oxidation than the control group, Ilmi rice, under salt stress.

For determination of characteristics for agronomic traits of T₃ and T₄ transgenic rice plant after sowing in Gunwi campus, development in a test field for transgenic plant of Kyungpook National University located Hyoryung-myun, Gunwi-gun, Gyeongsangbuk-do, South Korea was performed in May, 2010. On Oct. 10, 2010, characteristics for each organ of T₃ and T₄ transgenic rice plant were determined, and as a result, it was found that T₃ transgenic rice plant has better characteristics for agronomic traits than the control group (Ilmi rice) (FIG. 14). Characteristics for T₄ transgenic rice plant were also determined Transgenic rice plants analyzed are as follows: control group (Ilmi rice), pHY101, pHY102, pHY103, pHY104, pHY105, and pHY106.

According to the characteristics analysis for each organ of the rice plant cultivated in the GMO test field, it was found that the transgenic rice plant has better growth and higher crop yield and biomass than Ilmi rice.

### Example 4

### Determination of Salt Resistance and Molecular and Biological Characteristics of Rice Plant with T-DNA Inserted in MDHAR and DHAR Gene

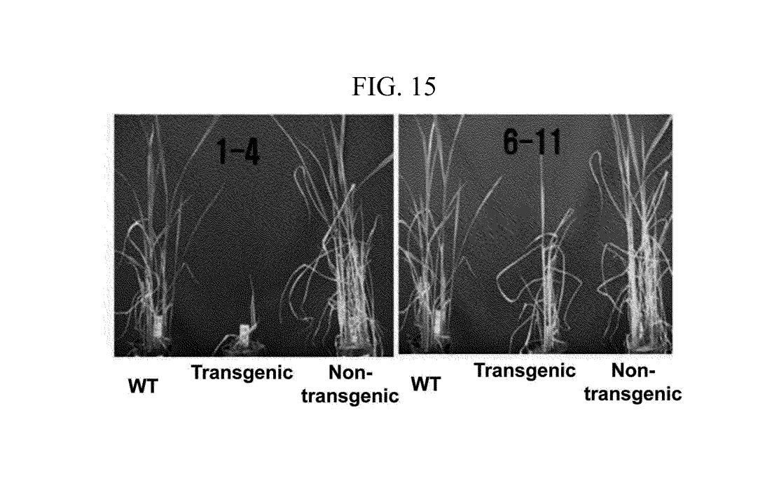

OsMdhar knock-down and OsDhar knock-out plant grown by sowing in a greenhouse and Gunwi campus on February and May, 2008 were treated with salt stress. As a result, it was found that the rice plant mutant with T-DNA insertion is more sensitive to salt stress than the control group, Dongjin rice. Treatment of 100 mM NaCl was performed for about 30 days. Based on the results, it was confirmed that the antioxidant genes play a very important role in plant growth in the presence and absence of salt stress (FIG. 15). #1-4 (OsMdhar) and #6-11 (OsDhar), which are a rice plant mutant with T-DNA insertion, were treated for about 30 days with 100 mM NaCl. It was found that each rice plant mutant with T-DNA insertion has higher sensitivity to salt stress than WT (Dongjin rice).

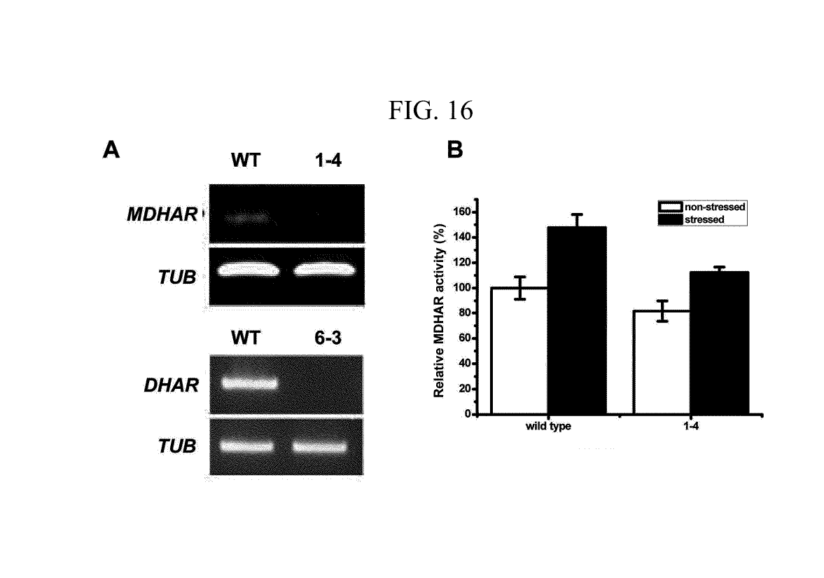

OsMdhar knock-down and OsDhar knock-out plant grown by sowing in a greenhouse and Gunwi campus on February and May, 2008 were treated with salt stress. A sample was collected in accordance with the treatment period, and RNA and protein were extracted for analysis of RNA expression and enzyme activity. In order to examine a decrease in gene expression of a rice plant mutant with T-DNA insertion, analysis of RNA expression was carried out by semi-quantitative RT-PCR. As a result, it was confirmed that the mRNA expression level of DHAR and MDHAR gene in the rice plant mutant with T-DNA insertion was lower than the control group, Dongjin rice (FIG. 16A), and each enzyme activity was also lowered in the rice plant mutant with T-DNA insertion than the control group, Dongjin rice (FIG. 16B). Based on the results, it was confirmed that the antioxidant gene of a rice plant plays a very important role in responding to salt stress condition.

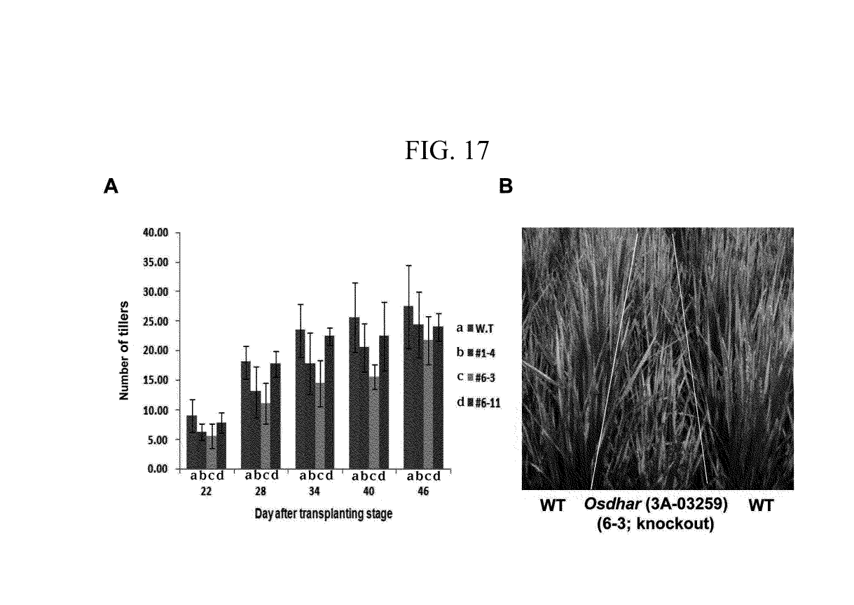

For measurement of the effective tiller number of OsMdhar knock-down and OsDhar knock-out plant after planting the rice plant mutant with T-DNA insertion in Gunwi campus, development in a GMO test field for transgenic plant of Kyungpook National University located Hyoryung-myun, Gunwi-gun, Gyeongsangbuk-do, South Korea was performed in June, 2009, and the effective tiller number was continuously observed from the planting. As a result, it was confirmed that the transgenic plant with T-DNA insertion has poorer initial tiller number than the control group (Dongjin rice). It was also found that the heading time lags behind Dongjin rice. Based on the result, it was found that the rice plant mutant with T-DNA insertion has poorer initial adaptability than Dongjin rice in a natural paddy field condition (FIG. 17). According to the determination of initial tiller number of the control group (Dongjin rice), #1-4 (OsMdhar), #6-3 (OsDhar), and #6-11 (OsDhar), it was found that the insertional mutant rice plant has less effective tiller number than the control group (Dongjin rice) (FIG. 17A), and the heading period was 10 to 20 days late in the insertional mutant rice plant than the control group, i.e., Dongjin rice (FIG. 17B).

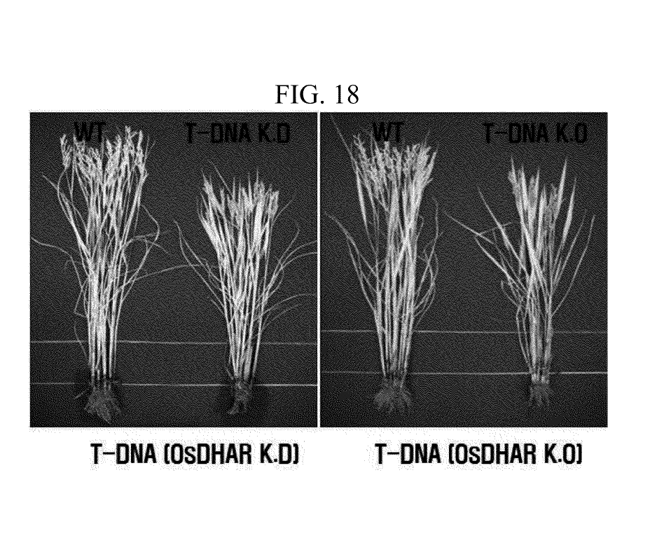

For morphology determination of OsDhar knock-out plant after planting the rice plant mutant with T-DNA insertion in Gunwi, development in a GMO test field for transgenic plant of Kyungpook National University located Hyoryung-myun, Gunwi-gun, Gyeongsangbuk-do, South Korea was performed in June, 2010, and the characters were continuously observed from the planting. As a result, it was confirmed that the rice plant mutant with T-DNA insertion has poorer early growth phase and late growth phase than the control group (Dongjin rice). Based on the result, it was found that the rice plant mutant with T-DNA insertion has poorer growth rate than Dongjin rice even in a common natural paddy field condition (FIG. 18). According to determination of growth state of the control group (Dongjin rice) and OsDhar, it was found that the rice plant mutant with T-DNA insertion has poorer growth state than the control group, Dongjin rice.

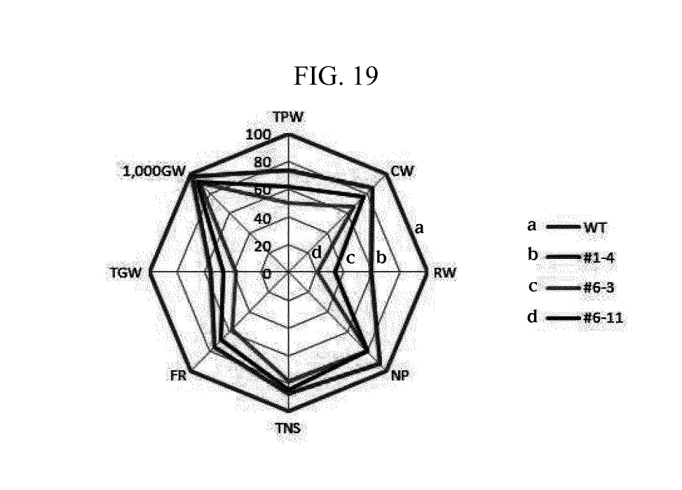

For determination of characteristics for each organ of the rice plant mutant with T-DNA insertion after planting in Gunwi campus, development in a GMO test field for transgenic plant of Kyungpook National University located Hyoryung-myun, Gunwi-gun, Gyeongsangbuk-do, South Korea was performed in June, 2010. On Oct. 10, 2010, characteristics for agronomic traits of the rice plant mutant with T-DNA insertion were determined, and as a result, it was found that the rice plant mutant with T-DNA insertion has poorer characteristics for each organ than the control group (Dongjin rice) (FIG. 19). Characteristics for agronomic traits were determined for the control group (Dongjin rice), #1-4 (OsMdhar), #6-3 (OsDhar), and #6-11 (OsDhar). According to the characteristics analysis for agronomic traits, it was found that the rice plant mutant with T-DNA insertion has much poorer characteristics for each organ compared to WT (Dongjin rice).

1. A method for increasing plant crop yield or enhancing resistance of a plant to environmental stress, the method comprising:
transforming a plant cell with a recombinant vector containing dhar (dehydroascorbate reductase) gene derived from rice (Oryza sativa) or mdhar (monodehydroascorbate reductase) derived from Chinese cabbage (Brassica rapa); and
overexpressing the dhar gene or mdhar gene.

2. The method according to claim 15, wherein the dhar gene or mdhar gene consists of the nucleotide sequence of SEQ ID NO: 1 and SEQ ID NO: 2, respectively.

3. A method for producing a plant with increased plant crop yield or enhanced resistance to environmental stress, the method comprising:
transforming a plant cell with a recombinant vector containing dhar (dehydroascorbate reductase) gene derived from rice (Oryza sativa) or mdhar (monodehydroascorbate reductase) derived from Chinese cabbage (Brassica rapa); and
regenerating a plant from the transformed plant cell.

4. A transgenic plant with increased crop yield that is produced by the method of claim 16.

5. A seed of the plant of claim 4.

6. (canceled)

7. The method according to claim 1, wherein the method is for enhancing resistance of the plant to environmental stress.

8. The method according to claim 7, wherein the dhar gene or mdhar gene consists of the nucleotide sequence of SEQ ID NO: 1 and SEQ ID NO: 2, respectively.

9. The method according to claim 7, wherein the environmental stress is salt, low temperature, or oxidative stress.

10. The method according to claim 3, wherein the method is for producing the transgenic plant with enhanced resistance to environmental stress.

11. The method according to claim 10, wherein the environmental stress is salt, low temperature, or oxidative stress.

12. A transgenic plant with enhanced resistance to environmental stress that is produced by the method of claim 10.

13. A seed of the plant of claim 12.

14. A composition for increasing crop yield or enhancing resistance of a plant to environmental stress, the composition comprising dhar (dehydroascorbate reductase) gene derived from rice (Oryza sativa) or mdhar (monodehydroascorbate reductase) derived from Chinese cabbage (Brassica rapa).

15. The method of claim 1, wherein the method is for increasing plant crop yield.

16. The method according to claim 3, wherein the method is for producing the plant with increased plant crop yield.
